(19) United States
(12) Patent Application Publication
(10) Pub. No.: US 2026/0034512 A1
(43) Pub. Date: Feb. 5, 2026

(54) AN APPARATUS FOR CATALYTIC DECOMPOSITION OF NITROUS OXIDE

(71) Applicant: MEDCLAIR AB, Stockholm (SE)

(72) Inventor: Olesya NIKONOVA, Uppsala (SE)

(73) Assignee: MEDCLAIR AB, Stockholm (SE)

(21) Appl. No.: 19/109,280

(22) PCT Filed: Sep. 5, 2023
(86) PCT No.: PCT/SE2023/050878

§ 371 Date: Mar. 6, 2025

(30) Foreign Application Priority Data
Sep. 12, 2022 (SE) 2251053-1

(51) Int. Cl. B01D 53/86; A61M 16/00; A61M 16/01

(52) U.S. Cl. CPC B01D 53/8631; A61M 16/009; A61M 16/01; A61M 2202/0283; B01D 2255/10; B01D 2255/2047; B01D 2255/20738; B01D 2255/20792; B01D 2257/402; B01D 2258/02

(57) ABSTRACT

An apparatus for catalytic decomposition of nitrous oxide in a gas stream derived from exhalation air from a patient. The apparatus comprises a casing housing a catalyst bed comprising a catalyst material promoting the decomposition of nitrous oxide. The casing comprises a gas inlet for a first gas stream derived from exhalation air from a patient. The casing comprises a gas outlets. The apparatus comprises one or more tubular members. The casing houses at least a portion of the tubular member. The tubular member comprises a gas inlet for a second gas stream comprising at least a portion of the first gas stream. The tubular member comprises a gas-permeable wall so as to provide one or more third gas streams from the tubular member to the catalyst bed. The third gas stream comprises at least a portion of the second gas stream.

### TECHNICAL FIELD

Aspects of the present invention relate to an apparatus for catalytic decomposition of nitrous oxide in a gas stream derived from exhalation air from a patient.

### BACKGROUND

Nitrous oxide, also known as laughing gas, is used in the medical field for pain relief. In general, mixtures of nitrous oxide (N₂O) and oxygen (O₂) are used. For example, nitrous oxide may be used in the fields of surgery, dental care and maternity care during delivery because of the anaesthetic and analgesic effects of nitrous oxide on a patient. In general, nitrous oxide is administered to the patient via a mask fitted over the nose and/or mouth of the patent.

In general, the composition of the air exhaled by a patient receiving nitrous oxide is substantially the same as the composition of the inhaled air except that there is an increase in moisture content (water) and carbon dioxide. In general, exhalation air from a patient inhaling nitrous oxide is collected and not released directly to the ambient so as to avoid exposure to members of the healthcare staff. Further, nitrous oxide is an air pollutant which is considered at least 300 times more effective than carbon dioxide as a “greenhouse gas”. Apparatuses using a catalyst material promoting the decomposition of nitrous oxide in the collected exhalation air may be used.

### SUMMARY

The inventor of the present invention has found drawbacks in conventional solutions for the decomposition of nitrous oxide in a gas stream derived from exhalation air from a patient. For example, some conventional solutions are not sufficiently efficient. For example, some conventional solutions involve a too complex design of the apparatus.

An object of the invention is to provide a solution which mitigates or solves drawbacks and problems of conventional solutions.

The above and further objects are solved by the subject matter of the independent claims. Further advantageous embodiments of the invention can be found in the dependent claims.

According to a first aspect of the invention, the above mentioned and other objects are achieved with an apparatus for catalytic decomposition of nitrous oxide in a gas stream derived from exhalation air from a patient, wherein the apparatus comprises
a casing housing one or more catalyst beds comprising a catalyst material promoting the decomposition of nitrous oxide,
wherein the casing comprises a gas inlet for a first gas stream derived from exhalation air from a patient,
wherein the casing comprises one or more gas outlets,
wherein the apparatus comprises one or more tubular members,
wherein the casing houses at least a portion of the tubular member,
wherein the tubular member comprises a gas inlet for a second gas stream comprising at least a portion of the first gas stream,
wherein the tubular member is configured to guide at least a portion of the second gas stream, and
wherein the tubular member comprises one or more gas-permeable walls so as to provide one or more third gas streams from the tubular member to the catalyst bed, the third gas stream comprising at least a portion of the second gas stream.

An advantage of the apparatus according to the first aspect is that the exhalation air from the patient more efficiently comes into contact with the catalyst material, or that that the catalyst material is more efficiently exposed to the exhalation air from the patient, whereby the decomposition of nitrous oxide is improved. An advantage of the apparatus according to the first aspect is that the exhalation air from the patient is evenly and efficiently spread, or distributed, to a major part of the catalyst bed or of the catalyst material. An advantage of the apparatus according to the first aspect is that the design of the apparatus can be made less complex in relation to conventional solutions, because a reduced or minimized pressure is required to guide the exhalation air into contact with the catalyst material. Thus, no additional equipment for actively applying a pressure to the gas stream derived from exhalation air is required. An advantage of the apparatus according to the first aspect is that the formation of hot spots in the catalyst material is avoided or minimized, inter alia because of the even and efficient guidance of the exhalation air to the catalyst material. Hot spots in the catalyst material may have an adverse effect on the catalyst material since the catalyst material may be destroyed, or deactivated, if the temperature is too high. An advantage of the apparatus according to the first aspect is that the decomposition of nitrous oxide in a gas stream derived from exhalation air from a patient is improved. The apparatus may be mobile, immobile, or stationary.

According to an advantageous embodiment of the apparatus according to the first aspect, the casing houses at least a portion of the gas-permeable wall of the tubular member. An advantage of this embodiment is that the design of the apparatus is even less complex in relation to conventional solutions. An advantage of this embodiment is that the decomposition of nitrous oxide in a gas stream derived from exhalation air from a patient is further improved.

According to a further advantageous embodiment of the apparatus according to the first aspect, the casing houses the gas-permeable wall of the tubular member, such as the entire gas-permeable wall of the tubular member. An advantage of this embodiment is that the exhalation air from the patient is even more efficiently spread to a major part of the catalyst material. An advantage of this embodiment is that the catalyst material is even more efficiently exposed to the exhalation air. An advantage of this embodiment is that the decomposition of nitrous oxide in a gas stream derived from exhalation air from a patient is further improved. An advantage of this embodiment is that the design of the apparatus can be made even less complex in relation to conventional solutions.

According to another advantageous embodiment of the apparatus according to the first aspect, the casing houses the tubular member, such as the entire tubular member. An advantage of this embodiment is that the exhalation air from the patient is even more efficiently spread to a major part of the catalyst material. An advantage of this embodiment is that the catalyst material is even more efficiently exposed to the exhalation air. An advantage of this embodiment is that the decomposition of nitrous oxide in a gas stream derived from exhalation air from a patient is further improved. An advantage of this embodiment is that the design of the apparatus can be made even less complex in relation to conventional solutions.

According to yet another advantageous embodiment of the apparatus according to the first aspect, the casing comprises one or more walls,
wherein the walls of the casing and of the one or more tubular members define a first space outside the tubular member, and
wherein the first space holds the catalyst bed.

An advantage of this embodiment is that the decomposition of nitrous oxide in a gas stream derived from exhalation air from a patient is further improved. An advantage of this embodiment is that the design of the apparatus can be made even less complex in relation to conventional solutions.

According to still another advantageous embodiment of the apparatus according to the first aspect, the tubular member defines a second space outside the first space, wherein the second space is free of any catalyst bed comprising a catalyst material. An advantage of this embodiment is that the decomposition of nitrous oxide in a gas stream derived from exhalation air from a patient is further improved.

According to an advantageous embodiment of the apparatus according to the first aspect, the apparatus comprises two or more tubular members, for example three or more tubular members. An advantage of this embodiment is that the exhalation air from the patient is even more efficiently spread to a major part of the catalyst material. An advantage of this embodiment is that the catalyst material is even more efficiently exposed to the exhalation air. An advantage of this embodiment is that the decomposition of nitrous oxide in a gas stream derived from exhalation air from a patient is further improved. An advantage of this embodiment is that the design of the apparatus can be made even less complex in relation to conventional solutions.

According to a further advantageous embodiment of the apparatus according to the first aspect, the apparatus comprises a frame holding the one or more tubular members. An advantage of this embodiment is that the design of the apparatus can be made even less complex in relation to conventional solutions. An advantage of this embodiment is that the decomposition of nitrous oxide in a gas stream derived from exhalation air from a patient is further improved.

According to another advantageous embodiment of the apparatus according to the first aspect, the casing houses the frame. An advantage of this embodiment is that the design of the apparatus can be made even less complex in relation to conventional solutions. An advantage of this embodiment is that the decomposition of nitrous oxide in a gas stream derived from exhalation air from a patient is further improved.

According to yet another advantageous embodiment of the apparatus according to the first aspect, the frame forms one or more through-holes for gas stream passage. An advantage of this embodiment is that the exhalation air from the patient is even more efficiently spread to a major part of the catalyst material. An advantage of this embodiment is that the catalyst material is even more efficiently exposed to the exhalation air. An advantage of this embodiment is that the decomposition of nitrous oxide in a gas stream derived from exhalation air from a patient is further improved. An advantage of this embodiment is that the design of the apparatus can be made even less complex in relation to conventional solutions.

According to still another advantageous embodiment of the apparatus according to the first aspect, the gas-permeable wall of the tubular member comprises one or more of the group of:
a grid; and
a mesh.

An advantage of this embodiment is that the design of the apparatus can be made even less complex in relation to conventional solutions. An advantage of this embodiment is that the decomposition of nitrous oxide in a gas stream derived from exhalation air from a patient is further improved.

According to an advantageous embodiment of the apparatus according to the first aspect, the catalyst bed comprises a catalyst material promoting direct decomposition of nitrous oxide.

According to a further advantageous embodiment of the apparatus according to the first aspect, the catalyst bed comprises solids of catalyst material promoting the decomposition of nitrous oxide, and wherein one or more spaces is/are formed between the solids. An advantage of this embodiment is that the exhalation air from the patient is even more efficiently distributed to a major part of the catalyst material. An advantage of this embodiment is that the catalyst material is even more efficiently exposed to the exhalation air. An advantage of this embodiment is that the decomposition of nitrous oxide in a gas stream derived from exhalation air from a patient is further improved. An advantage of this embodiment is that the design of the apparatus can be made even less complex in relation to conventional solutions.

According to another advantageous embodiment of the apparatus according to the first aspect, the tubular member comprises a first end portion and a second end portion,
wherein the tubular member has a longitudinal extension extending from the first end portion of the tubular member to the second end portion of the tubular member, and
wherein the first end portion of the tubular member comprises the gas inlet for the second gas stream.

An advantage of this embodiment is that the exhalation air from the patient is even more efficiently spread to a major part of the catalyst material. An advantage of this embodiment is that the catalyst material is even more efficiently exposed to the exhalation air. An advantage of this embodiment is that the decomposition of nitrous oxide in a gas stream derived from exhalation air from a patient is further improved. An advantage of this embodiment is that the design of the apparatus can be made even less complex in relation to conventional solutions.

According to yet another advantageous embodiment of the apparatus according to the first aspect, the apparatus comprises a base for resting on one or more horizontal surfaces, wherein the first end portion of the tubular member is located between the base and the second end portion of the tubular member. An advantage of this embodiment is that the longitudinal extension of the tubular member may extend in a substantially vertical direction with the gas inlet at the lower end when the base rests on a horizontal surface. An advantage of this embodiment is that the pressure required to guide the exhalation air through or into contact with the catalyst material can be further reduced or further minimized. An advantage of this embodiment is that the exhalation air from the patient is even more efficiently spread to a major part of the catalyst material. An advantage of this embodiment is that the catalyst material is even more efficiently exposed to the exhalation air. An advantage of this embodiment is that the decomposition of nitrous oxide in a gas stream derived from exhalation air from a patient is further improved. An advantage of this embodiment is that the design of the apparatus can be made even less complex in relation to conventional solutions.

According to still another advantageous embodiment of the apparatus according to the first aspect, the second end portion of the tubular member is tapered. An advantage of this embodiment is that the exhalation air from the patient is even more efficiently spread to a major part of the catalyst material. An advantage of this embodiment is that the catalyst material is even more efficiently exposed to the exhalation air. An advantage of this embodiment is that the decomposition of nitrous oxide in a gas stream derived from exhalation air from a patient is further improved.

According to an advantageous embodiment of the apparatus according to the first aspect, the casing comprises a first end portion and a second end portion,
wherein the casing has a longitudinal extension extending from the first end portion of the casing to the second end portion of the casing, and
wherein the apparatus comprises a base for resting on one or more horizontal surfaces, and
wherein the first end portion of the casing is located between the base and the second end portion of the casing.

An advantage of this embodiment is that the longitudinal extension of the casing may extend in a substantially vertical direction when the base rests on a horizontal surface. An advantage of this embodiment is that the decomposition of nitrous oxide in a gas stream derived from exhalation air from a patient is further improved.

According to a further advantageous embodiment of the apparatus according to the first aspect, the first end portion of the casing comprises the gas inlet for the first gas stream. An advantage of this embodiment is that the pressure required to guide the exhalation air through or into contact with the catalyst material can be further reduced or further minimized. An advantage of this embodiment is that the exhalation air from the patient is even more efficiently spread to a major part of the catalyst material. An advantage of this embodiment is that the catalyst material is even more efficiently exposed to the exhalation air. An advantage of this embodiment is that the decomposition of nitrous oxide in a gas stream derived from exhalation air from a patient is further improved. An advantage of this embodiment is that the design of the apparatus can be made even less complex in relation to conventional solutions.

According to another advantageous embodiment of the apparatus according to the first aspect, the longitudinal extension of the casing extends in a first direction, wherein the longitudinal extension of the tubular member extends in a second direction parallel to the first direction. An advantage of this embodiment is that the exhalation air from the patient is even more efficiently distributed to a major part of the catalyst material. An advantage of this embodiment is that the catalyst material is even more efficiently exposed to the exhalation air. An advantage of this embodiment is that the decomposition of nitrous oxide in a gas stream derived from exhalation air from a patient is further improved.

According to yet another advantageous embodiment of the apparatus according to the first aspect, the tubular member is detachably attached to the casing. An advantage of this embodiment is that the tubular member may be exchanged in an efficient manner. An advantage of this embodiment is that the decomposition of nitrous oxide in a gas stream derived from exhalation air from a patient is further improved.

According to still another advantageous embodiment of the apparatus according to the first aspect, the catalyst material comprises or consist of a noble metal or a combination of a noble metal with one of the group of: zinc; magnesium; and iron. An advantage of this embodiment is that the decomposition of nitrous oxide in a gas stream derived from exhalation air from a patient is further improved. For some embodiments, one or more of the noble metal, zinc, magnesium and iron may be loaded on aluminium oxide, titanium oxide, or on a zeolite support. For some embodiments, the catalyst material may comprise or consist of Al₂O₃. However, other types of catalyst material are possible.

According to an advantageous embodiment of the apparatus according to the first aspect, the apparatus comprises a heater for heating the catalyst material. An advantage of this embodiment is that the decomposition of nitrous oxide in a gas stream derived from exhalation air from a patient is further improved.

According to a further advantageous embodiment of the apparatus according to the first aspect, the catalyst bed surrounds the tubular member. An advantage of this embodiment is that the exhalation air from the patient is even more efficiently distributed to a major part of the catalyst material. An advantage of this embodiment is that the catalyst material is even more efficiently exposed to the exhalation air. An advantage of this embodiment is that the decomposition of nitrous oxide in a gas stream derived from exhalation air from a patient is further improved.

According to another advantageous embodiment of the apparatus according to the first aspect, the catalyst material is configured to promote the decomposition of nitrous oxide to nitrogen and oxygen.

The above-mentioned features and embodiments of the apparatus may be combined in various possible ways providing further advantageous embodiments.

According to a second aspect of the invention, the above mentioned and other objects are achieved with method for catalytic decomposition of nitrous oxide in a gas stream, which is derived from exhalation air from a patient, in an apparatus comprising a casing housing one or more catalyst beds comprising a catalyst material promoting the decomposition of nitrous oxide,
wherein the casing comprises a gas inlet for a first gas stream derived from exhalation air from a patient,
wherein the casing comprises one or more gas outlets,
wherein the apparatus comprises one or more tubular members,
wherein the casing houses at least a portion of the tubular member,
wherein the tubular member comprises a gas inlet for a second gas stream comprising at least a portion of the first gas stream,
wherein the tubular member comprises one or more gas-permeable walls, wherein the method comprises:
receiving the second gas stream via the gas inlet for the second gas stream;
guiding at least a portion of the second gas stream in the tubular member; and
providing one or more third gas streams from the tubular member to the catalyst bed through the one or more gas-permeable walls, the third gas stream comprising at least a portion of the second gas stream.

Advantages of the method according to the second aspect may correspond to advantages of the apparatus according to the first aspect and its embodiments mentioned above or below.

Further advantageous embodiments of the apparatus according to the first aspect and of the method according to the second aspect and further advantages with the embodiments of the present invention emerge from the detailed description of embodiments.

### BRIEF DESCRIPTION OF THE DRAWINGS

Embodiments of the invention will now be illustrated, for exemplary purposes, in more detail by way of embodiments and with reference to the enclosed drawings, where similar references are used for similar parts, in which:

FIG. 1 is a schematic sectional side view of an embodiment of the apparatus according to the first aspect of the invention;

FIG. 2 schematically illustrates a section of the apparatus along A-A in FIG. 1;

FIG. 3 is a schematic side view of the tubular members of FIG. 1;

FIG. 4 is a schematic perspective view of the tubular members of FIG. 1; and

FIG. 5 is a schematic flow chart illustrating aspects of embodiments of the method according to the second aspect of the invention.

### DETAILED DESCRIPTION

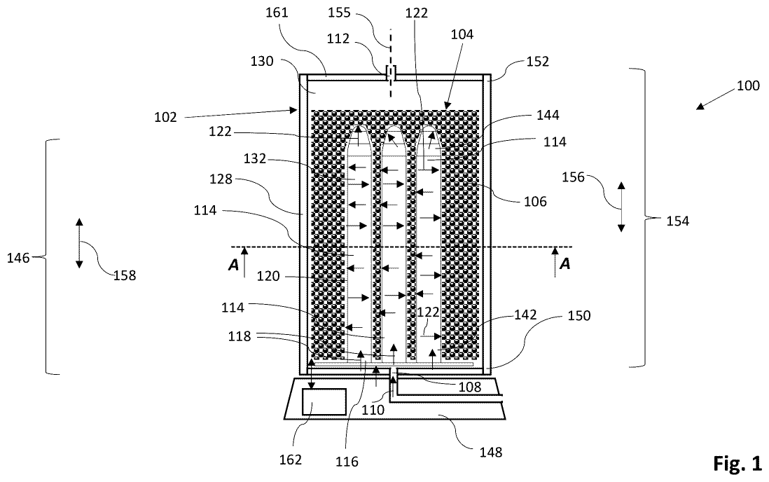

With reference to FIGS. 1 to 4, an embodiment of the apparatus 100 and aspects of embodiments of the apparatus 100 for catalytic decomposition of nitrous oxide in a gas stream derived from exhalation air from a patient, such as for direct catalytic decomposition of nitrous oxide, according to the first aspect of the invention are schematically illustrated. By direct catalytic decomposition of nitrous oxide is meant that the nitrous oxide is decomposed in one step, such into nitrogen (N₂) and oxygen (O₂), not in several steps, and not via any intermediate compositions. The apparatus 100 may be configured for direct catalytic decomposition of nitrous oxide into nitrogen (N₂) and oxygen (O₂) without using any reduction or oxidative substances. The apparatus 100 may be configured for direct catalytic decomposition without any reduction process. The apparatus 100 may be configured for direct catalytic decomposition without any formation of NOx. For some embodiments, the catalytic decomposition of nitrous oxide may be referred to as a catalytic decomposition reaction of nitrous oxide.

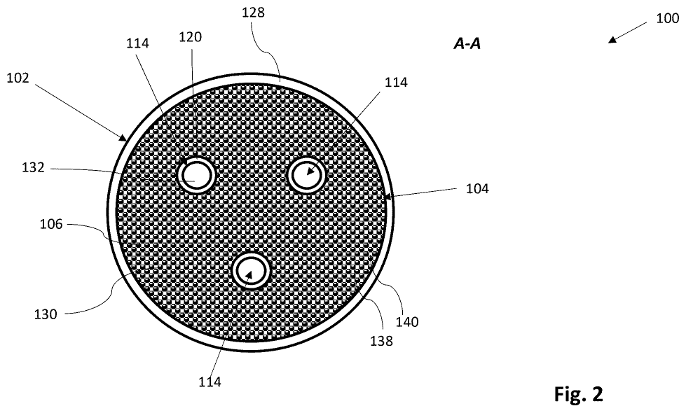

With reference to FIGS. 1 and 2, the apparatus 100 includes a casing 102 housing one or more catalyst beds 104, or one or more catalyst fixed beds. The catalyst bed 104 comprises a catalyst material 106 promoting the decomposition of nitrous oxide, such as the catalytic decomposition of nitrous oxide. For some embodiments, the catalyst bed 104 may be described to include a catalyst material promoting direct catalytic decomposition of nitrous oxide. For some embodiments, the catalyst material 106 may comprise or consist of a noble metal. For some embodiments, the catalyst material 106 may comprise or consist of a combination of a noble metal with one of the group of: zinc; magnesium; and iron. For some embodiments, one or more of the noble metal, zinc, magnesium and iron may be loaded on aluminium oxide, titanium oxide, or on a zeolite support. For some embodiments, the catalyst material may comprise or consist of Al₂O₃. However, other types of catalyst material are possible. For some embodiments, it may be defined that the catalyst material 106 is configured to promote the decomposition of nitrous oxide to nitrogen (N₂) and oxygen (O₂). For some embodiments, the catalyst material 106 does not have to be regenerated. Instead, the decomposition reaction may occur on the surface of the catalyst material 106, for example on the surface of a catalyst material 106 comprising or consisting of Al₂O₃.

With reference to FIG. 1, the casing 102 includes a gas inlet 108 for receiving a first gas stream 110 derived from exhalation air from a patient and including nitrous oxide. The casing 102 includes one or more gas outlets 112. For example, a mask for administering nitrous oxide to a patient and for capturing exhalation air from the patient may be fluidly connected to the gas inlet 108 of the casing 102 and may be mechanically connected to the gas inlet 108 of the casing 102, for example via one or more tubes or lines. Thus, for some embodiments, the apparatus 100 and/or the gas inlet 108 of the casing 102 may be connectable to a mask for administering nitrous oxide to a patient and for capturing exhalation air from the patient.

With reference to FIGS. 1 to 4, the apparatus 100 includes one or more tubular members 114. The apparatus 100 may comprise two or more tubular members 114, for example three or more tubular members 114. In the embodiment illustrated in FIGS. 1 to 4, the apparatus 100 has three tubular members 114. However, the apparatus 100 may have fewer than three tubular members 114, or more than three tubular members 114. The tubular member 114 may be described to be hollow. For some embodiments, the catalyst bed 104 may be described to surround the tubular member 114.

With reference to FIGS. 1 and 2, the casing 102 houses at least a portion of the tubular member 114. For some embodiments, it may be defined that the casing 102 houses the tubular member, such as the entire tubular member 114.

With reference to FIGS. 1 to 4, the tubular member 114 includes, or forms, a gas inlet 116 for receiving a second gas stream 118. The second gas stream 118 comprises at least a portion of the first gas stream 110, or the entire first gas stream 110. The tubular member 114 is configured to guide at least a portion of the second gas stream 118. The tubular member 114 includes one or more gas-permeable walls 120 so as to provide one or more third gas streams 122, for example several third gas streams 122, from the tubular member 114 to the catalyst bed 106, more specially one or more third gas streams 122 from the tubular member 114 to the catalyst bed 106 through the one or more gas-permeable walls 120. Thus, the gas-permeable wall 120 is permeable to gas. i.e. the gas-permeable wall 120 is configured to allow gas to pass. The third gas stream 122 comprises at least a portion of the second gas stream 118. For some embodiments, it may be defined that the gas outlet 112 of the casing 102 is configured to discharge one or more fourth gas streams.

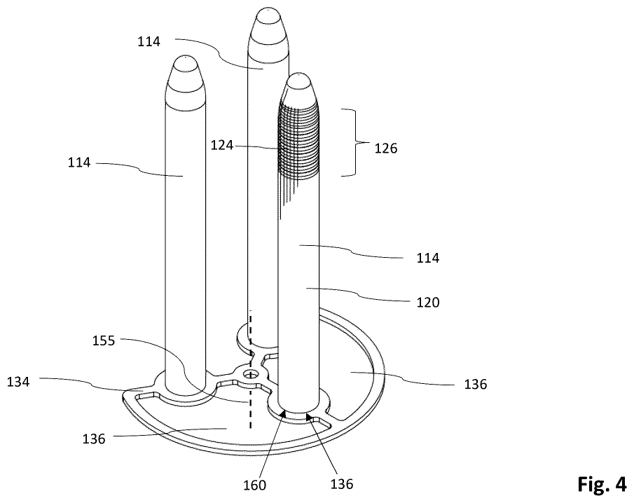

With reference to FIGS. 1 to 4, for some embodiments, it may be defined that the casing 102 houses at least a portion of the gas-permeable wall 120 of the tubular member 114. For some embodiments, it may be defined that the casing 102 houses the gas-permeable wall 120 of the tubular member 114, such as the entire gas-permeable wall 120 of the tubular member 114. For some embodiments, the gas-permeable wall 120 of the tubular member may include one or more of the group of: a grid; a net; and a mesh 124. In FIG. 4, only a section 126 of the gas-permeable wall 120 of the tubular member 114 is schematically illustrated to include a mesh 124. However, it is to be understood that a larger portion of the gas-permeable wall 120, a major portion of the gas-permeable wall 120, or the entire gas-permeable wall 120 of the tubular members 114 in FIG. 4 may include said mesh 124. It is to be understood that the gas-permeable wall 120 may include other gas-permeable members, elements or surfaces, such as a solid wall forming several through-holes, for example a perforated plate. For some embodiments, the tubular member 114 may include two or more gas-permeable walls 120 spaced apart from one another, for example spaced apart by one or more solid or non-gas-permeable walls, or spacers, for example spaced apart along a longitudinal extension 146 of the tubular member 114.

With reference to FIGS. 1 and 2, the casing 102 may include one or more walls 128. The walls 120, 128 of the casing 102 and of the one or more tubular members 114 may be described to define a first space 130 outside the tubular member 114. The first space 130 may hold, or contain, the catalyst bed 104, or the major portion of the catalyst bed 104. The tubular member 114 may be described to define a second space 132 outside the first space 130. The second space 132 is substantially free of any catalyst bed comprising a catalyst material, such as free of catalyst material, or substantially free of catalyst material.

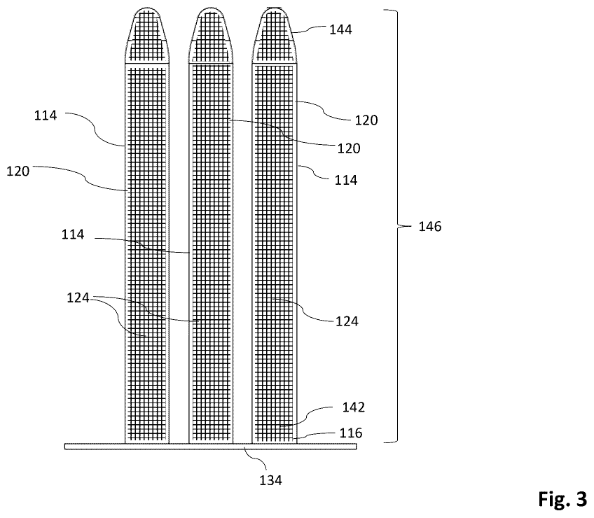

With reference to FIGS. 3 and 4, for some embodiments, the apparatus 100 may include a frame 134 holding the one or more tubular members 114. The casing 102 may house the frame 134. The frame 134 may form one or more through-holes 136 for gas stream passage.

With reference to FIGS. 1 and 2, the catalyst bed 104 may include solids 138 of catalyst material 106 promoting the decomposition of nitrous oxide, wherein one or more spaces 140 for gas flow is/are formed between the solids 138. The solid 138 may comprise a ball, a sphere, or a cylinder, or the solid 138 may have any other shape providing spaces 140 between the solids 138.

With reference to FIGS. 1 to 4, the tubular member 114 may include a first end portion 142 and a second end portion 144. The tubular member 114 may have a longitudinal extension 146 extending from the first end portion 142 of the tubular member 114 to the second end portion 144 of the tubular member 114. The first end portion 142 of the tubular member 114 may include the gas inlet 116 for the second gas stream 118. For some embodiments, the apparatus 100 may include a base 148 for resting on one or more horizontal surfaces. The first end portion 142 of the tubular member 114 may be located between the base 148 and the second end portion 144 of the tubular member 114. Thus, when the base 148 rests on a horizontal surface, the longitudinal extension 146 of the tubular member 114 may extend in a substantially vertical direction with the gas inlet 116 for the second gas stream 118 at the bottom end portion 142. The second end portion 144 of the tubular member 114 may be tapered. For some embodiments, the longitudinal extension 146 of the tubular member 114 may be in the range of 10 to 20 cm, for example 14 to 16 cm. For some embodiments, the outer diameter of the tubular member 114 may be in range of 1 to 3 cm, for example 1 to 2 cm.

With reference to FIGS. 1 and 2, the casing 102 may include a first end portion 150 and a second end portion 152. The casing 102 may have a longitudinal extension 154 extending from the first end portion 150 of the casing 102 to the second end portion 152 of the casing 102. The first end portion 150 of the casing 102 may be located between the base 148 and the second end portion 152 of the casing 102. Thus, when the base 148 rests on a horizontal surface, the longitudinal extension 154 of the casing 102 may extend in a substantially vertical direction. For some embodiments, the first end portion 150 of the casing 102 may include the gas inlet 108 for the first gas stream 110. Thus, when the first end portion 150 of the casing 102 includes the gas inlet 108 for the first gas stream 110 and when the base 148 rests on a horizontal surface, the longitudinal extension 154 of the casing 102 may extend in a substantially vertical direction with the gas inlet 108 for the first gas stream 110 at the bottom end portion 150. The second end portion 152 of the casing 102 may comprise one or more of the one or more gas outlets 112. The casing 102 may be attached, or attachable, to the base 148. For example, a mask for administering nitrous oxide to a patient and for capturing exhalation air from the patient may be mechanically connected, or connectable, to the base 148, for example, inter alia, via one or more tubes or lines. Thus, for some embodiments, the base 148 may be connectable to a mask for administering nitrous oxide to a patient and for capturing exhalation air from the patient. For some embodiments, the longitudinal extension 154 of the casing 102 may be in the range of 20 to 25 cm. For some embodiments, the outer diameter of the casing 102 may be in range of 7 to 15 cm, for example 8 to 12 cm. For some embodiments, the tubular members 114 may be evenly distributed about a longitudinal central axis 155 surrounded by the wall 128 of the casing 102.

With reference to FIG. 1, the longitudinal extension 154 of the casing 102 may extend in a first direction 156, wherein the longitudinal extension 146 of the tubular member 114 may extend in a second direction 158 parallel to the first direction 156.

With reference to FIG. 4, the tubular member 114 may be detachably attached to the casing 102, for example by way of the frame 134. For example, the frame 134 may form a seat 160 (see FIG. 4) or opening 136 for holding the tubular member 114. For example, the the tubular member 114 may be snapped into position in the seat 160 and may be held by the seat 160 by a snap-in locking arrangement, a friction locking arrangement, or a positive locking arrangement. However, the tubular member 114 may be attached to the casing 102 or to the frame 134 in other ways and by other locking arrangements.

With reference to FIG. 1, the casing 102 may comprise a cover 161 or top, which may be detachable or removable from the remainder of the casing 102, for example so as to provide access to the tubular member 114 or the catalyst bed 104 if needed, for example to a user or operator. The cover 161 may be detachably attached to the wall 128 of the casing 102.

With reference to FIG. 1, the apparatus 100 may include a heater 162 for heating the catalyst material 106. The heater 162 may comprise a heat exchanger. The heater 162 may be configured to heat the catalyst material 106 up to about 500° C., such as up to 520° C., or higher. One or more of the tubular member 114 and casing 102 may be made of material resistant to temperatures up to 600° C. One or more of the tubular member 114 and casing 102 may be made of material comprising or consisting of a metal or metal alloy, such as steel. The tubular member 114 and/or the casing 102 may be made of material comprising or consisting of a polymer or a polymer composite.

The apparatus 100 and/or casing 102 may be mobile and easily movable, or stationary. The apparatus 100 may be movable by a user or operator. The apparatus 100 may be directly connectable to at least one patient and/or to a mask for administering nitrous oxide to a patient and for capturing exhalation air from the patient. The apparatus 100 may be configured to be used or applied in the proximity of a patient receiving pain relief by inhaling oxygen containing nitrous oxide, also known as laughing gas. For some embodiments, the casing 102 and the catalyst bed 104 may be referred to as a decomposition reactor, or be part of a decomposition reactor. The apparatus 100 may include one or more temperature sensors for measuring the temperature of the catalyst bed 104. The apparatus 100 may comprise one or more gas analysers, for example of IR type, for measuring the concentration of nitrous oxide in one or more of the gas streams 110, 118, 122 mentioned above.

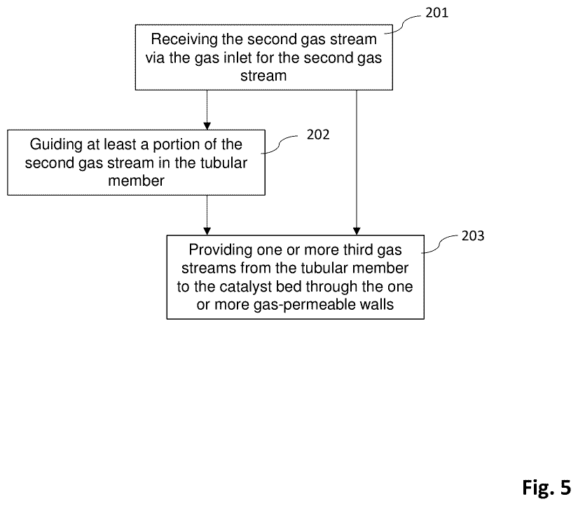

With reference to FIG. 5, aspects of embodiments of the method for catalytic decomposition of nitrous oxide in a gas stream, which is derived from exhalation air from a patient, in an apparatus 100 according to the second aspect of the invention are schematically illustrated in a flow chart. The apparatus 100 comprises a casing 102 housing one or more catalyst beds 104 comprising a catalyst material 106 promoting the decomposition of nitrous oxide,
wherein the casing 102 comprises a gas inlet 108 for a first gas stream 110 derived from exhalation air from a patient,
wherein the casing 102 comprises one or more gas outlets 112,
wherein the apparatus 100 comprises one or more tubular members 114,
wherein the casing 102 houses at least a portion of the tubular member 114,
wherein the tubular member 114 comprises a gas inlet 116 for a second gas stream 118 comprising at least a portion of the first gas stream 110,
wherein the tubular member 114 comprises one or more gas-permeable walls 120, wherein the method comprises:
receiving 201 the second gas stream 118 via the gas inlet 116 for the second gas stream 118;
guiding 202 at least a portion of the second gas stream 118 in, or inside, the tubular member 114; and
providing 203 one or more third gas streams 122 from the tubular member 114 to the catalyst bed 104 through the one or more gas-permeable walls 120, the third gas stream 122 comprising at least a portion of the second gas stream 118.

When an item is disclosed to be connected to another item in this disclosure, it may imply that the two items are fluidly connected to one another. When an item is disclosed to be connected to another item in this disclosure, it may imply that the two items are mechanically connected to one another. When an item is disclosed to be connected to another item in this disclosure, it may imply that the two items are both fluidly and mechanically connected to one another.

The present invention is not limited to the above-described embodiments. Instead, the present invention relates to, and encompasses all different embodiments being included within the scope of the appended independent claims.

1. An apparatus for catalytic decomposition of nitrous oxide in a gas stream derived from exhalation air from a patient, wherein the apparatus comprises
a casing housing one or more catalyst beds comprising a catalyst material promoting the decomposition of nitrous oxide,
wherein the casing comprises a gas inlet for a first gas stream derived from exhalation air from a patient,
wherein the casing comprises one or more gas outlets,
wherein the apparatus comprises one or more tubular members,
wherein the casing houses at least a portion of the tubular member,
wherein the tubular member comprises a gas inlet for a second gas stream comprising at least a portion of the first gas stream,
wherein the tubular member is configured to guide at least a portion of the second gas stream, and
wherein the tubular member comprises one or more gas-permeable walls so as to provide one or more third gas streams from the tubular member to the catalyst bed, the third gas stream comprising at least a portion of the second gas stream.

2. The apparatus according to claim 1, wherein the casing houses at least a portion of the gas-permeable wall of the tubular member.

3. The apparatus according to claim 1, wherein the casing houses the gas-permeable wall of the tubular member.

4. (canceled)

5. The apparatus according to claim 1, wherein the casing comprises one or more walls,
wherein the walls of the casing and of the one or more tubular members define a first space outside the tubular member, and
wherein the first space holds the catalyst bed.

6. The apparatus according to claim 1, wherein the tubular member defines a second space outside the first space, and
wherein the second space is free of any catalyst bed comprising a catalyst material.

7. The apparatus according to claim 1, wherein the apparatus comprises two or more tubular members.

8. (canceled)

9. (canceled)

10. (canceled)

11. The apparatus according to claim 1, wherein the gas-permeable wall of the tubular member comprises one or more of the group of:
a grid; and
a mesh.

12. The apparatus according to claim 1, wherein the catalyst bed comprises a catalyst material promoting direct decomposition of nitrous oxide.

13. The apparatus according to claim 1, wherein the catalyst bed comprises solids of catalyst material promoting the decomposition of nitrous oxide, and wherein one or more spaces is/are formed between the solids.

14. The apparatus according to claim 1, wherein the tubular member comprises a first end portion and a second end portion,
wherein the tubular member has a longitudinal extension extending from the first end portion of the tubular member to the second end portion of the tubular member, and
wherein the first end portion of the tubular member comprises the gas inlet for the second gas stream.

15. The apparatus according to claim 14, wherein the apparatus comprises a base for resting on one or more horizontal surfaces, and
wherein the first end portion of the tubular member is located between the base and the second end portion of the tubular member.

16. The apparatus according to claim 14, wherein the second end portion of the tubular member is tapered.

17. The apparatus according to claim 1, wherein the casing comprises a first end portion and a second end portion,
wherein the casing has a longitudinal extension extending from the first end portion of the casing to the second end portion of the casing, and
wherein the apparatus comprises a base for resting on one or more horizontal surfaces, and
wherein the first end portion of the casing is located between the base and the second end portion of the casing.

18. The apparatus according to claim 17, wherein the first end portion of the casing comprises the gas inlet for the first gas stream.

19. The apparatus according to claim 17, wherein the longitudinal extension of the casing extends in a first direction, and
wherein the longitudinal extension of the tubular member extends in a second direction parallel to the first direction.

20. The apparatus according to claim 1, wherein the tubular member is detachably attached to the casing.

21. The apparatus according to claim 1, wherein the catalyst material comprises or consist of a noble metal or a combination of a noble metal with one of the group of: zinc; magnesium; and iron.

22. The apparatus according to claim 1, wherein the apparatus comprises a heater for heating the catalyst material.

23. (canceled)

24. The apparatus according to claim 1, wherein the catalyst material is configured to promote the decomposition of nitrous oxide to nitrogen and oxygen.

25. A method for catalytic decomposition of nitrous oxide in a gas stream, which is derived from exhalation air from a patient, in an apparatus comprising a casing housing one or more catalyst beds comprising a catalyst material promoting the decomposition of nitrous oxide,
wherein the casing comprises a gas inlet for a first gas stream derived from exhalation air from a patient,
wherein the casing comprises one or more gas outlets,
wherein the apparatus comprises one or more tubular members,
wherein the casing houses at least a portion of the tubular member,
wherein the tubular member comprises a gas inlet for a second gas stream comprising at least a portion of the first gas stream,
wherein the tubular member comprises one or more gas-permeable walls, wherein the method comprises:
receiving the second gas stream via the gas inlet for the second gas stream;
guiding at least a portion of the second gas stream in the tubular member; and
providing one or more third gas streams from the tubular member to the catalyst bed through the one or more gas-permeable walls, the third gas stream comprising at least a portion of the second gas stream.
